(19) United States
(12) Patent Application Publication
(10) Pub. No.: US 2003/0099943 A1
(43) Pub. Date: May 29, 2003

(54) Diagnostic use of polymorphisms in the gene coding for the TNF receptor II and method for detecting non-responders to anti-TNF therapy

(75) Inventors: Stefan Schreiber, Kiel (DE); Jochen Hampe, Berlin (DE); Silvia Mascheretti, Kiel (DE)

Correspondence Address: James J. Murphy, Winstead Sechrest & Minick P.C., P.O. Box 50784, 1201 Main Street, Dallas, TX, 75250-0784, US

(73) Assignee: Conaris Research Institute Gmbh, Kiel (DE)

(21) Appl. No.: 09/902,176

(22) Filed: Jul. 10, 2001

(30) Foreign Application Priority Data
Jul. 10, 2000 (EP) 00114786.7

(51) Int. Cl. C12Q 1/68

(52) U.S. Cl. 435/6

(57) ABSTRACT

The invention relates to a method for detecting non-responders to anti-TNF therapy comprising testing an individual for homozygosity for a single nucleotid polymorphism in the gene coding for the TNF Receptor II. Monoclonal antibodies against TNF-α (infliximab) represent a new treatment for steroid refractory Crohn's disease that result in a remission rate of 30-50% after 4 weeks. Known single nucleotid polymorphisms within the TNF Receptor I and TNF Receptor II were tested for association with the response to the therapy. It was found that individuals homozygote for the mutated allele arginine at amino acid position +196 in the TNF Receptor II or the mutated allele in exon 2 at amino acid position 56 did not respond. Polymorphisms in exon 2 was newly found. None of the individuals homozygote for the mutations in exons 2 or 6 responded. The mutation in exon 2, although a silent mutation, can be used as a marker because it is in a high linkage disequilibrium with the mutation in exon 6.

The present invention relates to a method for detecting non-responders to anti-TNF therapy, the use of a novel polymorphism in exon 2 and a known polymorphism in exon 6 in the gene coding for the TNF Receptor II in anti-TNF therapy, to the genes containing the polymorphism in exon 2 or exons 2 and 6, and to the peptides encoded by the respective genes.

Crohn's disease is a chronic inflammatory disorder of the intestine. It shares many clinical and pathophysiological characteristics with other autoimmune disorders including rheumatoid arthritis. A polygenic aetiology of Crohn's disease is strongly suspected (Hugo, J. P. et al. Mapping of a susceptibility locus for Crohn's disease on chromosome 16. Nature 379, 821-823 (1996); Cho, J. H. et al. Identification of novel susceptibility loci for inflammatory bowel disease on chromosome 1p. 3q. and 4q: Evidence for epistasis between 1p and IBD1. PNAS 95, 7502-7505 (1998); Satsangi J. et al. Two stage genome-wide search in inflammatory bowel disease provides evidence for susceptibility loci on chromosome 3, 7 and 12. Nature Genet. 14, 188-202 (1996); Hampe J. et al. A genomewide analysis provides evidence for novel linkages in inflammatory bowel disease in a large European cohort, Am J Hum Genet 64, 808-816 (1999)); Hampe J. et al. Linkage of Inflammatory bowel disease to human chromosome 6p. Am J Hum Genet 65, 1647-1655 (1999), although disease genes have not been identified yet.

Glucocorticoids are an effective short term treatment of acute relapse in most patients. However, long term maintenance of remission is difficult in many patients. It is estimated that at least 50% of patients develop a steroid refractory and dependent disease (Munkholm P., Langholz E., Davisen M, Binder V. Frequency of glucocorticoid resistance and dependency in Crohn's disease. Gut 35, 360-362). An increased production of pro inflammatory cytokines including TNF-α (tumor necrosis factor α) in the intestinal mucosa is pivotal for the development of inflammatory relapses (Schreiber S. et al. Tumor necrosis factor alpha and interleukin 1 beta in relapse of Crohn's disease. Lancet 353, 459-461 (1999)) as well as chronic inflammatory activity.

The introduction of biological agents targeting TNF-α has led to impressive clinical results in therapy of refractory Crohn's disease.

Infliximab is a monoclonal antibody against TNF-α which was recently approved for therapeutic use in refractory and/or fistulating Crohn's disease in both the United States and Europe. Further, CDP571 and D2E7 are monoclonal antibodies directed against TNF-α with different biological properties, which have either been engineered from murine antibody genes or were generated by the phage-display system, respectively. In addition, recombinant TNF-receptor based proteins have been developed (e.g. ethanercept). All bind specifically to human TNF-α (not TNF-β) but vary in their murine parts as well as the human subclass used. It is unclear whether other mechanisms in addition to neutralization of TNF-α contribute to the therapeutic effect. It appears likely that at least infliximab can bind receptor attached or membrane expressed TNF-α and leads to deletion of activated immune cells either by complement activation or induction of apoptosis (Scallon B. J., Moore M. A., Trinh H., Knight D. M., Ghrayeb J., Chimeric anti-TNF-alpha monoclonal antibody cA2 binds recombinant transmembrane TNF-alpha and activates immune effector functions. Cytokine 7, 251-259 (1995)).

TNF Receptor I (CD120a) (Smith C. A., Farrah T., Goodwin R. G., Cell 76, 959-962 (1994); Baker S. J., Reddy E. P., Oncogene 12, 1-9 (1996)) is a 55/60 kDa (455 aa residues) transmembrane glycoprotein expressed in all nucleated mammalian cells. TNF Receptor II (CD120b) is a 75/80 kDa (461 aa residues) transmembrane glycoprotein expressed primarily by cells of the hematopoietic lineage and signals thymocyte and peripheral T-cells proliferation, natural killer cell and neutrophil activation. TNF Receptor II function is not completely known. Interaction between TNF-α and TNF Receptor II leads to a slow oligomerization of receptor molecules and ligand dissociation seems to occur before receptor-signalling complex formation. Membrane bound TNF-α seems to represent the effective ligand of TNF Receptor II. The mature human TNF Receptor II is a N and O glycosylated transmembrane protein.

The gene coding for the TNF Receptor II (SEQ ID NO: 49) is located on chromosome 1p36 and consists of 10 exons and 9 introns (Santee S. M. and Owen-Schaub L. B. Human Tumor Necrosis Factor Receptor p75/80 (CD120b) gene structure and promoter characterization. Journal Biological Chemistry 271;21151-21159 (1996)). Comparison of TNF Receptor II sequences obtained by different groups has identified six potential single nucleotide polymorphisms (SNPs) in exon 4, exon 6, exon 9 and exon 10 (Pantelidis P., Lympany P. A., Foley P. J., Fanning G. C. Welsh K. I. du Bois R. M. Polymorphic analysis of the high-affinity tumor necrosis factor receptor 2. Tissue Antigens 64: 585-591). In exon 4 nucleotide substitutions at position 511-512 (all nucleotide positions refer to cDNA to mRNA sequence of GeneBank accession number M32315) give rise to an arginine to proline substitution at aa position 143; in exon 6 nucleotide substitution at position 676 corresponds to a metionine to arginine substitution at aa position 196; in exon 9 nucleotide substitution at position 1176 creates an alanine to threonine change at aa position 365; finally, the nucleotide substitution at positions 1663, 1668 and 1690 in the 3′ untranslated region of exon 10. In addition to SNPs, were identified a (GATA)ₙ tetrameric repeat and a (GAA)(GGA) trimeric repeat in intron 1 (Santee S. M. and Owen-Schaub L. B. Human Tumor Necrosis Factor Receptor p75/80 (CD120b) gene structure and promoter characterization, Journal Biological Chemistry 271: 21151-21159 (1996)) and a (CA)₁₆ repeat in intron 4.

Previous studies suggest that polymorphisms in exons 6 and 10 of the gene coding for the TNF Receptor II, and amino acid exchange in TNF Receptor II potentially associated therewith, play a role in certain autoimmune diseases.

While polymorphisms in exon 4 and 9 have not been replicated, the ones in exon 6 and 10 have been studied in relation to several autoimmune diseases. Polymorphisms in exon 10 (3′UNR) at nucleotide positions 1663 and 1668 were tested in 90 patients with insulin dependent diabetes mellitus (IDDM), 101 with Graves' disease (GD) and 70 German healthy controls using Single Strand Conformation Polymorphism (SSCP) analysis (Rau H., Donner H., Usadel H. Badenhoop K. Polymorphisms of tumor necrosis factor receptor 2 are not associated with insulin-dependent diabetes mellitus or Graves' disease. Tissue antigens 49: 535-536 (1997)). Only one of the 2 polymorphic sites revealed 2 different alleles and no association was observed either with IDDM or GD in this German population. Contrasting results have been obtained for the coding mutation Met196Arg in exon 6 in relation to autoimmune diseases such as systemic Lupus erythematosus (SLE) and rheumatoid arthritis (RA). Met196Arg has been found not to be associated with RA in a Japanese population of 545 patients and 265 healthy controls (Shibue T. et al. Tumor necrosis factor alpha 5′ flanking region, tumor necrosis factor receptor II, and HLA-DRB1 polymorphisms in Japanese patients with rheumatoid arthritis. Tissue Antigens 43(4): 753-757 (2000) Rutgeerts Gastroenterolog 1999; 117: 761-69). In another Japanese population of 81 patients and 207 normal controls Arg196Met has been found associated with SLE (Komata T., Tsuchiya N., Matsushita M., Hagiwara K., Tokunaga K. Association of tumor necrosis factor receptor 2 (TNFR2) polymorphisms with susceptibility to systemic lupus erythematosus. Tissue Antigens 53: 527-533) but not in a cohort of 128 Spanish patients and 141 controls and in 74 UK patients and 90 controls (A1-Ansari A. S., Ollier W. E. R., Villarreal J., Ordl J. Teh L. S., Hajeer A. H. Tumor necrosis factor receptor II (TNFRII) exon 6 polymorphism in systemic lupus erythematosus. Tissue Antigens 55: 97-99 (2000)).

As regards Crohn's disease, a previous study in 193 Crohn's disease patients and 93 controls had suggested that polymorphism at position −308 in the TNF promoter might be related to disease localization and steroid dependency (Luis E. et al. Tumor necrosis factor (TNF) gene polymorphism in Crohn's disease (CD): influence on disease behaviour. Clinical and Experimental Immunology 199(1): 64-68 (2000)). The microsatellite allele TNFa2 has been found associated with TNF-α and -β secretion in human mononuclear cells (Pociot et al. Association of tumor necrosis factor (TNF) and class II major histocompatibility complex alleles with the secretion of TNF-a and TNF-b by human mononuclear cells: a possible link to insulin-dependent diabetes mellitus. Eur J Immunol 23: 224-231 (1993)).

It has been outlined above that a new therapy against Crohn's disease involving the use of monoclonal antibodies directed against TNF-α has been developed. A one time infusion of the monoclonal antibody infliximab (commercially available as Remicade®) at a dose of 5-20 mg/kg bodyweight results in a remission rate of approximately 30-40% without statistical differences between dose groups (Targan S. R. et al. A short-term study of chimeric monoclonal antibody cA2 to tumor necrosis factor alpha for Crohn's disease. Crohn's disease cA2 Study Group. N Engl J Med 337, 1029-1035 (1997)). Although intensely investigated, clinical parameters (e.g. disease activity, which are related to the height of mucosal TNF-α production (Reinecker et al. Enhanced secretion of tumor necrosis factor-alpha, IL-6 and IL-1 by isolae lamina propria mononuclear cells from patients with ulcerative colitis and Crohn's disease, Clin Exp Immunol 94, 174-181 (1993)) could not be identified as predictors for responsiveness. Non-response appears to be a stable characteristic with patients staying non-responsive even if consecutive infusions are applied. The duration of response to a single dose anti TNF-α is variable with symptoms recurring in most patients after 6-12 weeks.

Infliximab infusions are generally well tolerated, although side effects resulting from intense immunosuppression have been described (including pneumonia, reactivation of intracellular infections, sepsis and abscess formation). In addition, 5 of about 600 patients (with rheumatoid arthritis and Crohn's disease) treated in clinical studies developed a malignant lymphoproliferative disease, and one more sporadic lymphoma has been reported in the more than 30,000 patients with Crohn's disease treated in the USA.

Taking into consideration that in only 30-40% of all patients receiving infliximab an alleviation of symptoms can be observed, it is a matter of fact that 60-70% of them receive infliximab without any therapeutic advantage but with the risk of potential severe side effects. Therefore, and also in view of the high price of biological therapeutics like infliximab, a possibility to predict, whether or not a specific patient suffering from Crohn's disease will respond to the therapy, would be highly desirable.

Therefore, it is an object of the present invention to provide a simple test for detecting non-responsiveness to anti-TNF therapy, in particular infliximab therapy, in a considerable percentage of non-responders.

It is a further object of the present invention to provide a polymorphism in a gene, which polymorphism, can be used for diagnostic purposes.

It is an additional object of the present invention to provide the use of a polymorphism in a gene for anti-TNF therapy or Crohn's disease.

It is also an object of the present invention to provide a genetic sequence containing at least one polymorphism rendering the gene suitable for diagnostic purposes.

The object is achieved by a method for detecting non-responders to anti-TNF therapy, comprising testing an individual for homozygosity for at least one single nucleotide polymorphism in the gene coding for the TNF Receptor II.

The object is also achieved by a novel single nucleotide polymorphism (SNP), a transition A to G, in position 257, or 168 from the transcription starting site, in exon 2 of the gene coding for the TNF Receptor II.

The object is further achieved by the use of the single nucleotide polymorphism (SNP), the transition T to G, in position 676, or 587 from the transcription starting site, in exon 6 of the gene coding for the TNF Receptor II.

The object is, in addition, achieved by the genes having the sequences identified in SEQ ID NO 51 and SEQ ID NO 53, and by nucleotide sequences coding for the same peptides or peptides having the same immunological properties, i. e. the same blocking and/or competing properties.

The SNP in exon 2 corresponds to a silent mutation at amino acid position 56 (Lys56Lys), and the SNP in exon 6 corresponds to an amino acid change at position 196 (Met196Arg). Coupling analysis reveals that both SNPs are in strong linkage disequilibrium. Therefore, the SNP in exon 2 can be used as a marker for the SNP in exon 6.

It is a great advantage of the present invention that it can be accomplished with DNA which can be derived from any cell, e.g. blood cells or other cells or body fluids, e.g. saliva, or other body parts. Preferably, DNA is derived from EDTA-blood.

It appeared likely to us that the differential response to infliximab represents a polygenic trait. Therefore, we decided to screen for mutations, by direct sequencing, the promoter, the 10 exons (including the 3′UNR) of the TNF Receptor II in 45 individuals of a study population consisting of 90 Crohn's disease patients and 180 controls, and then test the mutations present in the population, as well as known mutations in the TNF Receptor II and Receptor I, in a cohort of 90 Crohn's disease patients receiving infliximab.

3 fragments of the 5′ regulatory region, the 10 exons and 1 fragment of the 3′ untranslated region of the TNF Receptor II were direct sequenced in 45 individuals of the study population; the same primers, designed on the basis of the published sequence (Santee and Owen-Schaub, 1996), were used for PCR Amplification and sequencing: first fragment of the 5′ regulatory region: forward primer 5′CTTCCACGAGGTGACATCTCC3′ (SEQ ID NO: 1), reverse primer 5′GCCCTAATACAGGGCCAGC3′ (SEQ ID NO: 2), second fragment: forward primer 5′GGACAGATTGCAGCTGGAATG3′ (SEQ ID NO: 3), reverse primer 5′TAGAGCCAGACCACCTGGGT3′ (SEQ ID NO: 4); third fragment: forward primer 5′AGCCTGGACAACATGGCGA3′ (SEQ ID NO: 5), reverse primer 5° CCCTCGACTGAAAGCGAAAG3′ (SEQ ID NO: 6); exon 1: forward primer (promoter) 5′GAGGCGTGTCCAAGGCC3′ (SEQ ID NO: 7), reverse primer (intron 1) 5′GCGCGGAGTCACCACCT3′ (SEQ ID NO: 8); exon 2: forward primer (intron 1) 5′ATCACCCATGGCAGAACCC3′ (SEQ ID NO: 9), reverse primer (intron 2) 5′TGCCCTCACCCGGC3′ (SEQ ID NO: 10); exon 3: forward primer (intron 2) 5′GACTCTGGCCTTGTTTCCTCA3′ (SEQ ID NO: 11), reverse primer (intron 3) 5′GGGAAGTTGGAGGCAGGG3′ (SEQ ID NO: 12); exon 4: forward primer (intron 3) 5′TGACCGTTTGCGCCCTC3′ (SEQ ID NO: 13), reverse primer (intron 4) 5′GTCCCCAAGGACCTGAGCC3′ (SEQ ID NO: 14); exons 5 and 6: forward primer (intron 4) 5′AGACAGAGCTCCTTGGGC3′ (SEQ ID NO: 15), reverse primer (intron 6) 5′GCAGACAGAAGCAGTGAATGA3′ (SEQ ID NO: 16); exons 7 and 8: forward primer (intron 6) 5′TCCTGGCTTGCTGGCTG3′ (SEQ ID NO: 17), reverse primer (intron 8) 5′GAGGGCAGTGGAGACAC3′ (SEQ ID NO: 18); exon 9: forward primer (intron 8) 5′GCTGACTGCTCTCCCCT3′ (SEQ ID NO: 19), reverse primer (intron 9) 5′TGGGAAGAAGCAGGTGTG3′ (SEQ ID NO: 20); exon 10: forward primer (intron 9) 5′GAATCTGCATCTTGGGCAGG3′ (SEQ ID NO: 21), reverse primer (3′ untranslated) 5′GAGGCTGCGGCTGTGGA3′ (SEQ ID NO: 22); 3′ untranslated region: forward primer 5′CGGTGTGGGCTGTGTCGTA3′ (SEQ ID NO: 23) and reverse primer 5′CCTACAGGGCTGCCACCTC3′ (SEQ ID NO: 24). Direct sequencing was conducted using BigDye Terminator (PE Biosystems) and run on an automated sequencer ABI 310 (PE Biosystems).

Direct sequencing of the 3 regions of the promoter and of the 10 exons of the TNF Receptor II confirmed the polymorphisms at amino acid position 196 (exon 6), at nucleotide position 1663, 1668 and 1690 in the 3′ UNR while the amino acid position 143 in exon 4 and the nucleotide positions −1413 and −1120 in the promoter did not appear polymorphic in the 45 individuals tested. The same applies to the polymorphism in exon 9. In addition, we identified a novel polymorphism, a transition (A to G) in the third codon position of amino acid 56 (Lysine) in exon 2 (nucleotide position 168 from the transcription starting site). This mutation appears to be in strong linkage disequilibrium with the Met196 Arg in exon 6. On the basis of the data available in the literature and the results of our own sequencing we therefore decided to test in the cohort of patients receiving infliximab the following mutations: TNF Receptor I at promoter position −609 and in exon 1 at nucleotide position 36, corresponding to amino acid 12 (silent mutation); and TNF Receptor II in exon 2, silent mutation at amino acid position 56, further in exon 6, amino acid change at position 196 and in the 3′ untranslated region in exon 10 at nucleotide position 1663 and 1690.

An open label, prospective multicenter clinical trial, which was conducted in 31 German centers, was specifically set up for the evaluation of pharmacogenomics and biological markers of response. Inclusion criteria (steroid/azathioprine refractory Crohn's disease, stable co-medication before and throughout the study) were similar to those used in previously published studies which established the clinical efficacy of infliximab in Crohn's disease (Present D.H. et al/Infliximab for the Treatment of Fistulas in Patients with Crohn's Disease N. Eng J Med 340(18): 1398-405 (1999); Targan S. R. et al. A short-term study of chimeric monoclonal antibody cA2 to tumor necrosis factor alpha for Crohn's disease. Crohn's disease cA2 Study Group. N Engl J Med 337, 1029-1035 (1997). The trial was conducted and monitored according to the standards of “Good Clinical Practice” (GCP). The protocol and the genetic test procedures received prior approval by all local ethics committees/institutional review boards. 96 patients with moderate to severe steroids refractory or steroid dependent Crohn's disease (10 mg or more/day) active for at least six months, with CDAI between 220 and 450, decided to participate in the treatment protocol and to provide EDTA (ethylene diamine tetraacetic acid) blood for DNA based analysis after written informed consent. At the time of the analysis clinical data was missing for 6 patients leaving the study cohort to 90 patients (55 women and 33 men and 2 unknown). The overall remission rate (as indicated by a Crohn's disease activity index below 150 points) (38% at 4 weeks) was in the range as expected by previous studies (Targan S. R. et al. A short-term study of chimeric monoclonal antibody cA2 to tumor necrosis factor alpha for Crohn's disease. Crohn's disease cA2 Study Group. N Engl J Med 337, 1029-1035 (1997); Schraub L. B. Human tumor necrosis factor receptor p75/80 (CD120b) gene structure and promoter characterisation. J. Biol. Chemistry 271, 21151-21159 (1996)). On enrolment in the trial EDTA blood was obtained from each patient as well as from 180 German blood donors as normal controls. DNA was extracted by standard techniques (e.g. using DNAzol based on a guanidine-detergent lysing solution) and dispensed on 96 well plates (20 ng/well).

6 SNPs were genotyped using TaqMan (ABI 7700 PE Biosystems, Foster City, Calif.) allelic discrimination: 2 SNPs in the TNF Receptor I gene (12p13), one in the promoter at position −609 from the transcription starting site and one in exon 1, a silent mutation at amino acid position 12, Pro12Pro (CCA-CCG) and 4 SNPs in the TNF Receptor II gene (1p36), a silent mutation in exon 2 at amino acid position 56, Lys56Lys (AAA-AAG), a second codon position in exon 6 changing amino acid 196, Met196Arg (ATG-AGG), and 2 mutations in exon 10 in the 3′ untranslated region at nucleotide position 1663 and 2007. Genotypes were assigned without knowledge of treatment response. Primers and probes (see table 1) were designed using Primer Express (PE Biosystems) and purchased from Eurogentec. PCR amplification was conducted with the termocycler 9700 (PE Biosystems) in a final volume of 10 μl. The amplification conditions involved two pre-PCR steps of 2 min at 50° C. and 10 min at 95° C. followed by a variable number of cycles including a denaturation step at 95° C. for 15 sec and an annealing step of 1 min at different temperatures for the different assays (see table 2).

While TaqMan allelic discrimination is an approved technique, the present invention should not be regarded as being limited thereto. The genotype may as well be determined by direct sequencing, RFLP (restriction fragment length polymorphism), PCR (polymerase chain reaction)—based techniques or any other technique or combination of techniques known to those skilled in the art for identifying a specific mutation.

A further particularly suitable procedure is PCR followed by restriction digestion with N1aIII ←5′CATGΛ3′ (commercially available from New England Biolabs with catalogue number #125S or #125L. This enzyme cuts at position 277, 677, 941 etc. Regarding the exon 6-polymorphism, primers between 278 and 940 will yield one cut in the wild type and no cut in the mutant.

PCR-SSCP with 3′ mismatches in forward and reverse primers was described in Pentelidis et al., Tissue antigens 54: 585-591 (1999) for the mutation in exon 6 as well as for exons 4, 9 and 10.

The genotypes obtained by TaqMan were checked by direct sequencing in 45 individuals of the study population obtaining in every case identical result.

TABLE 1
TaqMan primers and probes:

TNF-R1 promoter-609 (G/T)

| | | |
|---|---|---|
| FAM probe (G allele) | 5′ACAGATCCAGACAGGTTCAGTTATGTGTCTGAGAAGTT3′ | (SEQ ID NO:25) |
| TET probe (T allele) | 5′ACAGATCCAGACAGTTTCAGTTATGTGTCTGAGAAGTT3′ | (SEQ ID NO:26) |
| Forward primer | 5′GACAGGTTATCTCCACTCTGCAAA3′ | (SEQ ID NO:27) |
| Reverse primer | 5′CAATTCAGAATGCTTAGCTTTTTAGC3′ | (SEQ ID NO:28) |

TNF-R1 Exon1 Pro12Pro (A/G)

| | | |
|---|---|---|
| FAM probe (G allele) | 5′TGCTGCTGCCGCTGGTGAGACC3′ | (SEQ ID NO:29) |
| TET probe (A allele) | 5′AACTGCTGCTGCCACTGGTGAGACC3′ | (SEQ ID NO:30) |
| Forward primer | 5′CTTGGGACGTCCTGGACAGAC3′ | (SEQ ID NO:31) |
| Reverse primer | 5′AAGGTGCCTCGCCCACC3′ | (SEQ ID NO:32) |

TNF-R2 Exon2 Lys56Lys (A/G)

| | | |
|---|---|---|
| FAM probe (A allele) | 5′TGCAGCAAATGCTCGCCGGGT3′ | (SEQ ID NO:33) |
| TET probe (G allele) | 5′TGCAGCAAGTGCTCGCCGGG3′ | (SEQ ID NO:34) |
| Forward primer | 5′CAGAGAATACTATGACCAGACAGCTCA3 | (SEQ ID NO:35) |
| Reverse primer | 5′GAGTGCCCCCGTGGCT3′ | (SEQ ID NO:36) |

TNF-R2 Exon6 Met196Arg (T/G)

| | | |
|---|---|---|
| FAM probe (T allele) | 5′AATGCAAGCATGGATGCAGTCTGCAC3′ | (SEQ ID NO:37) |
| TET probe (G allele) | 5′AATGCAAGCAGGGATGCAGTCTGCAC3′ | (SEQ ID NO:38) |
| Forward primer | 5′GCTGTAACGTGGTGGCCATC3′ | (SEQ ID NO:39) |
| Reverse primer | 5′CTGGGTTCTGGAGTT3′ | (SEQ ID NO:40) |

TNF-R2 3′UNT nt 1663 (G/A)

| | | |
|---|---|---|
| FAM probe (A allele) | 5′AGAGGCAGCGAGTTGTGGAAAGCCTC3′ | (SEQ ID NO:41) |
| TET probe (G allele) | 5′AGGCAGCGGGTTGTGGAAAGCCT3′ | (SEQ ID NO:42) |
| Forward primer | 5′ACCACTAGGACTCTGAGGCTCTTTC3′ | (SEQ ID NO:43) |
| Reverse primer | 5′CCAGCCAGCCTTCCGAG3′ | (SEQ ID NO:44) |

TNF-R2 3′UNT nt 1690 (T/C)

| | | |
|---|---|---|
| FAM probe (C allele) | 5′CCTCTGCTGCCATGGCGTGTCC3′ | (SEQ ID NO:45) |
| TET probe (T allele) | 5′CCTCTGCTGCCATGGTGTGTCCT3′ | (SEQ ID NO:46) |
| Forward primer | 5′CTGCAGGCCAAGAGCAGAG3′ | (SEQ ID NO:47) |
| Reverse primer | 5′GGTTTTCTGGAAGCCAGAGCT3′ | (SEQ ID NO:48) |

TABLE 2
Probes and primers concentrations and amplification conditions
for TaqMan assays (ABI 7700).

| | | | | | Annealing | |
|---|---|---|---|---|---|---|
| | FAM | | Forward | Reverse | temperature | N. of |
| SNP position | Probe | TET Probe | Primer | Primer | and time | cycles |
| TNF-R1 Promoter-609 | 200 nM | 200 nM | 300 nM | 300 nM | 1 min 64° C. | 55 |
| TNF-R1 Exon 1 Pro12Pro | 200 nM | 200 nM | 300 nM | 300 nM | 1 min 60° C. | 50 |
| TNF-R2 Exon 2 Lys56Lys | 200 nM | 200 nM | 900 nM | 900 nM | 1 min 60° C. | 40 |
| TNF-R2 Exon 6Met196Arg | 100 nM | 100 nM | 50 nM | 900 nM | 1 min 62° C. | 50 |
| TNF-R2 3′UNT nt 1663 | 200 nM | 200 nM | 300 nM | 300 nM | 1 min 60° C. | 50 |
| TNF-R2 3′UNT nt 1690 | 200 nM | 200 nM | 50 nM | 900 nM | 1 min 60° C. | 50 |

Of the 6 mutations tested only the one in exon 6 of the TNF Receptor II leads to an amino acid exchange (Met→Arg at amino acid position 196). Exon 6 codes a small portion of the transmembrane region and part of the extracellular domain including the proteolytic cleavage site that produces the soluble form of TNF Receptor II. Amino acid 196 is located within the extracellular region near one of the two N-glycosylation sites. It appears to be positioned at the border of the area for which the receptor structure can be predicted and it may have a possible influence on receptor conformation (Zimmer, Lengauer, personal communication). Presently, it is not certain whether this mutation has a functional significance.

The allele and genotype frequency in the controls were comparable to the ones obtained by Ansari in Spanish and UK populations outlined above. Within our patient population, for the mutation in exon 6 there were 61 (67.8%) homozygote wild type, 23 (25.5%) heterozygote and 6 (6.7%) homozygote mutant. For the mutation in exon 2 there were 58 (64.4%) homozygote wild type, 25 (27.8%) heterozygote and 7 (7.8%) homozygote mutant (table 3).

TABLE 3
Genotype and allele frequency of the Met196Arg among the 90
infliximab treated patients and 180 controls (from sex matched healthy
blood donors recruited from the German population).

| | Patients (total 90) | Controls (total 180) |
|---|---|---|
| Genotype frequency | | |
| Met196 (wild type) | 0.678 (61) | 0.672 (121) |
| Met196Arg (heterozygote) | 0.255 (23) | 0.300 (54) |
| Arg196 (mutant) | 0.067 (6) | 0.028 (5) |
| Allele frequency | | |
| Metl96 (wild type) | 0.805 | 0.821 |
| Arg106 (mutant) | 0.195 | 0.179 |

TaqMan results were checked by direct sequencing in 45 individuals.

It was found that homozygocity for the single nucleotide polymorphism in exon 6 is always associated with non-response to infliximab (i.e. neither reaching clinical improvement (drop of the CDAI by at least 70 points) nor remission (CDAI<150 points) resulting in a test specificity of 100% in these individuals (table 4). Homozygote individuals show a marked reduction in clinical improvement (as indicated by a significantly smaller drop in the Crohn's disease activity index) after treatment with infliximab whereas a heterozygous genotype was not associated with an altered clinical response (table 4), i.e. the observed differences between homozygote and heterozygote individuals were at a statistically not significant level. The single nucleotide polymorphism at amino acid 196 (exon 6) of TNF Receptor II leads to a non-conservative amino acid substitution between Met(ATG) and Arg(AGG). About 10% of non-responders are characterized by this genetic variation.

TABLE 4
Distribution of exon 6 genotype frequency among responders
and not responders after 4 weeks from the infiximab infusion. The
response has been evaluated as reduction of CDAI of 70 points, 100
points and as clinical remission (CDAI less than 150 points)

| | Response | | Response | | | |
|---|---|---|---|---|---|---|
| | 70 points | | 100 points | | | |
| | (after | | (after | | Clinical remission | |
| | 4 weeks) | | 4 weeks) | | (after 4 weeks) | |
| | Yes | No | Yes | No | Yes | No |
| Met196 (wild type) | 36 | 25 | 32 | 29 | 18 | 42 |
| 61 patients | | | | | | 1 not known |
| Heterozygote | 17 | 6 | 13 | 10 | 11 | 12 |
| 23 patients | | | | | | |
| Arg 196 (mutant) | 0 | 6 | 0 | 6 | 0 | 6 |
| 6 patients | | | | | | |

The Crohn's disease activity index incorporates 8 variables related to the disease activity: the number of liquid or very soft stools, the severity of abdominal pain or cramping, general well-being, the presence of extra-intestinal manifestations, abdominal mass, use of antidiarrheal drugs, haematocrit and body weight. These items yield a composite score ranging from 0 to approximately 650. Higher scores indicate greater disease activity. Scores below 150 are compatible with remission, whereas scores above 550 indicate severe illness.

By testing patients suffering from Crohn's disease before treatment with infliximab for the mutation in exon 6, at least 10% of those which will not respond can be detected in advance and be excluded from the useless therapy.

A second mutation in the same gene, the silent mutation in exon 2, is in a high degree of linkage disequilibrium, i.e. in almost complete linkage disequilibrium (4 discordant genotypes out of 90, i.e. there was 1 genotype heterozygote for the mutation in exon 6 and homozygote mutant for the mutation in exon 2, and 3 genotypes homozygote wild type for the mutation in exon 6 and heterozygote for the mutation in exon 2.) with the polymorphism in exon 6. Again, homozygotes are completely non-responsive to anti-TNF treatment with infliximab.

Therefore, the mutation in exon 2 can be used as a marker, to detect the same non-responders as the test for the exon 2 polymorphism.

In the TNF Receptor I gene no mutations were detected with an influence on the amino acid sequence (table 5). None of the mutations was in linkage disequilibrium with the mutation in exon 6 of the TNF Receptor II and no association with a therapeutic response was seen.

TABLE 5
Mutations tested and gene localisation

| Gene and chromosomal | Locailsation and characteristics of the |
|---|---|
| localisation | mutations: |
| TNF Receptor I (p55) | Promoter −609 |
| chromosome 12p13*** | Pro12Pro (Pro CCA-CCG) (nucleotide |
| ** | position +36 MspA1, extracellular |
| | domain) |
| TNF Receptor II (p75) | Lys56Lys (exon 2) |
| chromosome 1p36 | Met196Arg (exon 6, extracellular domain) |
| | 2 mutations in the 3′UNT region |

**(Weinshenker et al., Neurology 52, 1500-1503 (1999)
***Fuchs Peter, Strehl Sabine, Dworzak Michael, Himmler Adolf and Ambors Peter Structure of the Human TNF Receptor 1 (p60) Gene (TNFR1) and Localisation to Chromosome 12p13. Genomics (1992) 13: 219-224

The effect of the polymorphism at position 168 in exon 2 has been shown exemplarily for Crohn's disease. The applicability of this polymorphism, however, is not restricted to Crohn's disease, but extends to any disease wherein TNF α plays a role, in particular inflammatory or malignant diseases.

1. A method for detecting non-responders to anti-TNF therapy, comprising testing an individual for homozygosity for at least one single nucleotide polymorphism in the gene coding for the TNF Receptor II.

2. The method of claim 1, wherein anti-TNF therapy is infliximab therapy.

3. The method of claim 1, wherein anti-TNF therapy is therapy of Crohn's disease.

4. The method of claim 2, wherein anti-TNF therapy is therapy of Crohn's disease.

5. The method of claim 1, wherein the at least one single nucleotide polymorphism is nucleotide substitution T/G at position 587 from the transcription starting site in exon 6 of the gene coding for the TNF Receptor II.

6. The method of claim 1, wherein the at least one single nucleotide polymorphism is nucleotide substitution A/G at position 168 from the transcription starting site in exon 2 of the gene coding for the TNF Receptor II.

7. The method of claim 5, comprising identifying the mutation T/G at position 587 by a technique suitable therefor.

8. The method of claim 6, comprising identifying the mutation A/G at position 168 by a technique suitable therefor.

9. The method of claim 1, comprising the use of blood cells for providing DNA.

10. Use of a polymorphism at position 168 (A/G) in exon 2 of the gene coding for the TNF Receptor II for diagnostic purposes.

11. The use of claim 10 in an inflammatory or malignant or other chronic disease.

12. The use of claim 11 in Crohn's disease.

13. The use of claims 10 in anti-TNF therapy.

14. Use of a polymorphism at position 587 (T/G) in exon 6 of the gene coding for the TNF Receptor II in Crohn's disease.

15. Use of a polymorphism at position 587 (T/G) in exon 6 of the gene coding for the TNF Receptor II in anti-TNF therapy.

16. A kit comprising reagents tailored to identify the polymorphism at position 168 (A/G) in exon 2 of the gene coding for the TNF-Receptor II.

17. A kit comprising reagents tailored to identify the polymorphism at position 587 (T/G) in exon 6 of the gene coding for the TNF-Receptor II.

18. A kit comprising reagents tailored to identify the polymorphism at position 168 (A/G) in exon 2 and the polymorphism at position 587 (T/G) in exon 6 of the gene coding for the TNF-Receptor II.

19. Gene having the nucleotide sequence identified in SEQ ID NO 51 or a nucleotide sequence coding for the same peptide or a peptide having the same immunological properties.

20. Gene having the nucleotide sequence identified in SEQ ID NO 53 or a nucleotide sequence coding for the same peptide or a peptide having the same immunological properties.

21. Peptide having the sequence identified in SEQ ID NO 52 or a peptide having the same immunological properties.

22. Peptide having the sequence identified in SEQ ID NO 54 or a peptide having the same immunological properties.


SEQUENCE LISTING (54 sequences)

SEQ ID NO: 1 - DNA, 21 bp, Artificial Sequence
  Description of Artificial Sequence Forward Primer
cttccacgaggtgacatctcc

SEQ ID NO: 2 - DNA, 19 bp, Artificial Sequence
  Description of Artificial Sequence Reverse Primer
gccctaatacagggccagc

SEQ ID NO: 3 - DNA, 21 bp, Artificial Sequence
  Description of Artificial Sequence Forward Primer
ggacagattgcagctggaatg

SEQ ID NO: 4 - DNA, 20 bp, Artificial Sequence
  Description of Artificial Sequence Reverse Primer
tagagccagaccacctgggt

SEQ ID NO: 5 - DNA, 19 bp, Artificial Sequence
  Description of Artificial Sequence Forward Primer
agcctggacaacatggcga

SEQ ID NO: 6 - DNA, 20 bp, Artificial Sequence
  Description of Artificial Sequence Reverse Primer
ccctcgactgaaagcgaaag

SEQ ID NO: 7 - DNA, 17 bp, Artificial Sequence
  Description of Artificial Sequence Forward Primer
gaggcgtgtccaaggcc

SEQ ID NO: 8 - DNA, 17 bp, Artificial Sequence
  Description of Artificial Sequence Reverse Primer
gcgcggagtcaccacct

SEQ ID NO: 9 - DNA, 19 bp, Artificial Sequence
  Description of Artificial Sequence Forward Primer
atcacccatggcagaaccc

SEQ ID NO: 10 - DNA, 14 bp, Artificial Sequence
  Description of Artificial Sequence Reverse Primer
tgccctcacccggc

SEQ ID NO: 11 - DNA, 21 bp, Artificial Sequence
  Description of Artificial Sequence Forward Primer
gactctggccttgtttcctca

SEQ ID NO: 12 - DNA, 18 bp, Artificial Sequence
  Description of Artificial Sequence Reverse Primer
gggaagttggaggcaggg

SEQ ID NO: 13 - DNA, 17 bp, Artificial Sequence
  Description of Artificial Sequence Forward Primer
tgaccgtttgcgccctc

SEQ ID NO: 14 - DNA, 19 bp, Artificial Sequence
  Description of Artificial Sequence Reverse Primer
gtccccaaggacctgagcc

SEQ ID NO: 15 - DNA, 18 bp, Artificial Sequence
  Description of Artificial Sequence Forward Primer
agacagagctccttgggc

SEQ ID NO: 16 - DNA, 21 bp, Artificial Sequence
  Description of Artificial Sequence Reverse Primer
gcagacagaaggagtgaatga

SEQ ID NO: 17 - DNA, 17 bp, Artificial Sequence
  Description of Artificial Sequence Forward Primer
tcctggcttgctggctg

SEQ ID NO: 18 - DNA, 17 bp, Artificial Sequence
  Description of Artificial Sequence Reverse Primer
gagggcagtggagacac

SEQ ID NO: 19 - DNA, 17 bp, Artificial Sequence
  Description of Artificial Sequence Forward Primer
gctgactgctctcccct

SEQ ID NO: 20 - DNA, 18 bp, Artificial Sequence
  Description of Artificial Sequence Reverse Primer
tgggaagaagcaggtgtg

SEQ ID NO: 21 - DNA, 20 bp, Artificial Sequence
  Description of Artificial Sequence Forward Primer
gaatctgcatcttgggcagg

SEQ ID NO: 22 - DNA, 17 bp, Artificial Sequence
  Description of Artificial Sequence Reverse Primer
gaggctgcggctgtgga

SEQ ID NO: 23 - DNA, 19 bp, Artificial Sequence
  Description of Artificial Sequence Forward Primer
cggtgtgggctgtgtcgta

SEQ ID NO: 24 - DNA, 19 bp, Artificial Sequence
  Description of Artificial Sequence Reverse Primer
cctacagggctgccacctc

SEQ ID NO: 25 - DNA, 38 bp, Artificial Sequence
  Description of Artificial Sequence FAM Probe
acagatccagacaggttcagttatgtgtctgagaagtt

SEQ ID NO: 26 - DNA, 38 bp, Artificial Sequence
  Description of Artificial Sequence TET Probe
acagatccagacagtttcagttatgtgtctgagaagtt

SEQ ID NO: 27 - DNA, 24 bp, Artificial Sequence
  Description of Artificial Sequence Forward Primer
gacaggttatctccactctgcaaa

SEQ ID NO: 28 - DNA, 26 bp, Artificial Sequence
  Description of Artificial Sequence Reverse Primer
caattcagaatgcttagctttttagc

SEQ ID NO: 29 - DNA, 22 bp, Artificial Sequence
  Description of Artificial Sequence FAM Probe
tgctgctgccgctggtgagacc

SEQ ID NO: 30 - DNA, 25 bp, Artificial Sequence
  Description of Artificial Sequence TET Probe
aactgctgctgccactggtgagacc

SEQ ID NO: 31 - DNA, 21 bp, Artificial Sequence
  Description of Artificial Sequence Forward Primer
cttgggacgtcctggacagac

SEQ ID NO: 32 - DNA, 17 bp, Artificial Sequence
  Description of Artificial Sequence Reverse Primer
aaggtgcctcgcccacc

SEQ ID NO: 33 - DNA, 21 bp, Artificial Sequence
  Description of Artificial Sequence FAM Probe
tgcagcaaatgctcgccgggt

SEQ ID NO: 34 - DNA, 20 bp, Artificial Sequence
  Description of Artificial Sequence TET Probe
tgcagcaagtgctcgccggg

SEQ ID NO: 35 - DNA, 27 bp, Artificial Sequence
  Description of Artificial Sequence Forward Primer
cagagaatactatgaccagacagctca

SEQ ID NO: 36 - DNA, 16 bp, Artificial Sequence
  Description of Artificial Sequence Reverse Primer
gagtgcccccgtggct

SEQ ID NO: 37 - DNA, 26 bp, Artificial Sequence
  Description of Artificial Sequence FAM Probe
aatgcaagcatggatgcagtctgcac

SEQ ID NO: 38 - DNA, 26 bp, Artificial Sequence
  Description of Artificial Sequence TET Probe
aatgcaagcagggatgcagtctgcac

SEQ ID NO: 39 - DNA, 20 bp, Artificial Sequence
  Description of Artificial Sequence Forward Primer
gctgtaacgtggtggccatc

SEQ ID NO: 40 - DNA, 15 bp, Artificial Sequence
  Description of Artificial Sequence Reverse Primer
ctgggttctggagtt

SEQ ID NO: 41 - DNA, 26 bp, Artificial Sequence
  Description of Artificial Sequence FAM Probe
agaggcagcgagttgtggaaagcctc

SEQ ID NO: 42 - DNA, 23 bp, Artificial Sequence
  Description of Artificial Sequence TET Probe
aggcagcgggttgtggaaagcct

SEQ ID NO: 43 - DNA, 25 bp, Artificial Sequence
  Description of Artificial Sequence Forward Primer
accactaggactctgaggctctttc

SEQ ID NO: 44 - DNA, 17 bp, Artificial Sequence
  Description of Artificial Sequence Reverse Primer
ccagccagccttccgag

SEQ ID NO: 45 - DNA, 22 bp, Artificial Sequence
  Description of Artificial Sequence FAM Probe
cctctgctgccatggcgtgtcc

SEQ ID NO: 46 - DNA, 23 bp, Artificial Sequence
  Description of Artificial Sequence TET Probe#
cctctgctgccatggtgtgtcct

SEQ ID NO: 47 - DNA, 19 bp, Artificial Sequence
  Description of Artificial Sequence Forward Primer
ctgcaggccaagagcagag

SEQ ID NO: 48 - DNA, 21 bp, Artificial Sequence
  Description of Artificial Sequence Reverse Primer
ggttttctggaagccagagct

SEQ ID NO: 49 - DNA, 3683 bp, Homo sapiens
  CDS (90)..(1475)
  mat_peptide (156)
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttggggaaagcctctgctgccatggtgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgcccagctctggcttccagaaaaccccagcatccttttctgcagaggggctttctggagaggagggatgctgcctgagtcacccatgaagacaggacagtgcttcagcctgaggctgagactgcgggatggtcctggggctctgtgtagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaaaagcaccgcctccaaatgctaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 50 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 51 - DNA, 3683 bp, Homo sapiens
  CDS (90)..(1475)
  mat_peptide (156)
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaagtgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttggggaaagcctctgctgccatggtgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgcccagctctggcttccagaaaaccccagcatccttttctgcagaggggctttctggagaggagggatgctgcctgagtcacccatgaagacaggacagtgcttcagcctgaggctgagactgcgggatggtcctggggctctgtgtagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaaaagcaccgcctccaaatgctaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 52 - protein, 461 aa, Homo sapiens; identical to SEQ ID NO: 50

SEQ ID NO: 53 - DNA, 3683 bp, Homo sapiens
  CDS (90)..(1475)
  mat_peptide (156)
gcgagcgcagcggagcctggagagaaggcgctgggctgcgagggcgcgagggcgcgagggcagggggcaaccggaccccgcccgcacccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaagtgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcagggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttggggaaagcctctgctgccatggtgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgcccagctctggcttccagaaaaccccagcatccttttctgcagaggggctttctggagaggagggatgctgcctgagtcacccatgaagacaggacagtgcttcagcctgaggctgagactgcgggatggtcctggggctctgtgtagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaaaagcaccgcctccaaatgctaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaaaaaaaa

SEQ ID NO: 54 - protein, 461 aa, Homo sapiens [sequence omitted]